(19) United States
(12) Patent Application Publication
(10) Pub. No.: US 2026/0041704 A1
(43) Pub. Date: Feb. 12, 2026

(54) COMPOSITIONS AND METHODS FOR TREATING SICKLE CELL DISEASE

(71) Applicant: Beren Therapeutics P.B.C., West Hollywood, CA (US)

(72) Inventors: Ian Pyrah, Bellevue, WA (US); Eric Niesor, Lyon (FR)

(21) Appl. No.: 19/099,986

(22) PCT Filed: Aug. 2, 2023
(86) PCT No.: PCT/IB2023/057837

§ 371 Date: Jan. 30, 2025

Related U.S. Application Data
(60) Provisional application No. 63/394,938, filed on Aug. 3, 2022.

(51) Int. Cl. A61K 31/724; A61P 7/00; A61P 7/06

(52) U.S. Cl. CPC A61K 31/724; A61P 7/00; A61P 7/06

(57) ABSTRACT

The present disclosure provides methods and compositions for treating or preventing a sickle cell disease (SCD). The SCD may include sickle cell anemia (SCA). The method may include administering a composition such as a cyclodextrin to a subject in need of SCD treatment. Some embodiments include contacting a RBC with a cyclodextrin.

### CROSS-REFERENCE

This application claims the benefit of U.S. provisional application No. 63/394,938, filed Aug. 3, 2022, which application is incorporated herein by reference.

### FIELD OF THE DISCLOSURE

The present disclosure relates to treatment or prevention of sickle cell disease (SCD), such as sickle cell anemia (SCA). In particular, the disclosure relates to treatment or prevention of sickle cell disease (SCD) by administration of a hydroxypropyl-beta-cyclodextrin (HPBCD).

### BACKGROUND

Sickle cell disease (SCD) affects over 50 million people, such as more than 100,000 people in the United States. It is estimated that 5% of the world population is affected, and few treatments are available. The most common type of SCD is sickle cell anemia (SCA). SCA significantly impairs quality of life and shortens lifespan. While hemolytic anemia and frequent vaso-occlusive crisis are the most frequent complications for patients suffering from SCA, these patients are also at very high risk for developing pulmonary hypertension, cerebral vasculopathy leading to stroke, osteonecrosis, retinopathy, priapism, leg ulcers, acute chest syndrome and glomerulopathy (Kato et al. Pediatr Hematol Oncol 24(3): 159-170 (2007)).

There is an unmet need for therapeutic agents to treat or prevent SCD, such as SCA.

Normally, red blood cells are disc-shaped and flexible enough to move easily through the blood vessels. Red blood cells affected by SCD may be crescent-or “sickle”-shaped. These cells may be rigid, and may not bend or move easily and hence can block blood flow to the rest of the body. The blocked blood flow through the body can lead to serious problems, including stroke, eye problems, infections and episodes of pain called pain crises.

Sickle cell anemia (SCA) is a common type of sickle cell disease (SCD) and may be the most frequent hemoglobinopathy in the world, affecting over 50 million people. It is estimated that 5% of the world population is affected by these hemoglobinopathies for which very few treatments are available. SCA is caused in many instances by a mutation in the B-globin gene, leading to production of a structurally abnormal hemoglobin, called hemoglobin S (HbS). Sickle Cell Disease patients may be homozygous for the mutated HbS gene (HbSS) referred to as SS and the healthy subjects may be homozygous for the wild type HbA gene (HbAA) referred to as AA. Sickle Cell Trait is defined as heterozygous for the HbS gene (HbSA). HbS may polymerize in deoxygenated conditions, causing red blood cell (RBC) sickling. Because of this feature, sickled RBCs are often more rigid, fragile and prone to hemolysis than healthy, normal RBCs.

The loss of RBC deformability may be a factor responsible for adverse effects in SCD patients. Further, a high concentration of cholesterol in RBC membranes may decrease its capacity to transport oxygen, which may trigger the sickling process. Increased RBC membrane cholesterol content even in a subject without SCD may affect the deformability and visco-elasticity properties of the cells. In addition, release of free hemoglobin in plasma caused by the lysis of fragile RBCs may also cause other problems in SCD patients.

SCD may be a lifelong disease with few treatment options. A blood or bone marrow transplant may currently be the only cure for SCD. Most sickle cell treatments are aimed at relieving symptoms or preventing complications. For example, antibiotics can be administered to prevent bacterial infections and sepsis. Blood transfusions may be given acutely to treat severe anemia, acute chest syndrome, or other life-threatening complications that may occur as part of SCD. Children with sickle cell disease may receive chronic blood transfusions to prevent complications such as strokes. Hydroxyurea therapy can be administered to cause the body to produce fetal hemoglobin, a type of hemoglobin normally found only in fetuses and the very young. Voxelotor can be administered to increase the affinity between hemoglobin and oxygen which can inhibit the sickling of the red blood cells.

Recently a gene therapy treatment has been developed for sickle cell disease. The treatment silences BOL11A. a gene that normally suppresses the production of fetal hemoglobin after birth. There is currently an ongoing Phase 2 clinical trial to determine if the gene therapy will lead to a complete absence of the vaso-occlusive events in severe SCD. However, gene therapy carries a risk of inducing mutations and thereby causing cancer. Thus, even with these available treatments there is a need for additional safe and effective treatments for sickle cell disease. It would be particularly desirable to provide a treatment for sickle cell disease which improves RBC deformability without making the RBC more susceptible to hemolysis.

### SUMMARY

Disclosed herein are methods and compositions useful for treatment of sickle cell disease (SCD). The methods reduce membrane cholesterol concentration, improve deformability, and/or reduce hemolysis of a red blood cell (RBC) of a subject suffering from or at risk for sickle cell disease (SCD). Disclosed herein are methods of reducing membrane cholesterol concentration, improving deformability, and/or reducing hemolysis of a red blood cell (RBC) of a subject suffering from or at risk for sickle cell disease (SCD), comprising contacting the RBC with an effective amount of a cyclodextrin. In some embodiments, the methods are methods of reducing membrane cholesterol concentration of a red blood cell (RBC) of a subject suffering from or at risk for Sickle Cell Disease (SCD), comprising contacting the RBC with an effective amount of a cyclodextrin. In some embodiments, contacting the RBC with an effective amount of a cyclodextrin comprises administering the effective amount of the cyclodextrin to the subject, wherein the subject comprises the RBC. Disclosed herein are methods of treating or preventing sickle cell disease (SCD) in a subject in need thereof, comprising administering an effective amount of a cyclodextrin. In some embodiments, the membrane cholesterol levels in red blood cells (RBCs) of the subject is at least 10%, 20%, 30%, 40%. 50%, 60%, 70%, 80%, 90%, 100%, 150%, 200%, 250%, 300%, 400%, 500%, or more, higher than the membrane cholesterol levels in red blood cells (RBCs) of a healthy subject. In some embodiments, the SCD comprises or is sickle cell anemia (SCA). In some embodiments, the subject is homozygous for a genetic mutation that results in the SCD. In some embodiments, the cyclodextrin comprises, or is, a beta (β)-cyclodextrin. In some embodiments, the β-cyclodextrin is a hydroxypropyl-β-cyclodextrin (HPBCD). In some embodiments, the HPBCD is a 2-hydroxypropyl-β-cyclodextrin (2-HPBCD). In some embodiments, the β-cyclodextrin comprises a mixture of 2-hydroxypropyl-β-cyclodextrin (2-HPBCD) molecules. In some embodiments, the mixture comprises at least 10% 2-HPBCD molecules selected from beta-cyclodextrin substituted with four hydroxypropyl groups, beta-cyclodextrin substituted with five hydroxypropyl groups, beta-cyclodextrin substituted with six hydroxypropyl groups, beta-cyclodextrin substituted with seven hydroxypropyl groups and beta-cyclodextrin substituted with eight hydroxypropyl groups. In some embodiments, the mixture comprises less than 1% beta-cyclodextrin substituted with one hydroxypropyl group and less than 1% unsubstituted beta-cyclodextrin. In some embodiments, the mixture comprises at least 10% beta-cyclodextrin substituted with four hydroxypropyl groups. In some embodiments, the mixture comprises at least 10% beta-cyclodextrin substituted with five hydroxypropyl groups. In some embodiments, the mixture comprises at least 10% beta-cyclodextrin substituted with six hydroxypropyl groups. In some embodiments, the mixture comprises at least 10% beta-cyclodextrin substituted with seven hydroxypropyl groups. In some embodiments, the mixture comprises at least 10% beta-cyclodextrin substituted with eight hydroxypropyl groups. In some embodiments, the hydroxypropyl-β-cyclodextrin (2-HPBCD) molecules in the mixture have an average degree of substitution of from about 3 to about 10. In some embodiments, the administration is intravenous. In some embodiments, before the administering step. a RBC of the subject exhibits increased membrane cholesterol levels or reduced deformability compared to a healthy subject. In some embodiments, after the administering step, a RBC of the subject exhibits decreased membrane cholesterol levels or increased deformability, compared to the subject before the administering step. In some embodiments, the method reduces membrane cholesterol levels in the subject. In some embodiments, the method maintains deformability or increases deformability of RBC of the subject. In some embodiments, the method increases RBC deformability in the subject to at least about 5%, at least about 10%, at least about 15%, about 10% to 20%, about 10% to 30%, about 10% to 40%, about 10% to 50%, about 10% to 60%, about 10% to 70%, about 10% to 80%, about 10% to 90% or about 10% to 100% of the deformability of RBCs of a healthy subject. In some embodiments, the method increases RBC deformability in the subject to at least about 5%, about 10%, about 15%, or about 10% to about 20% of the deformability of RBCs of a healthy subject. In some embodiments, after the administering step, the deformability of a RBC of the subject is at least about 1.1 fold, about 1.2 fold, about 1.3 fold, about 1.4 fold, about 1.5 fold, about 2 fold, about 3 fold, or about 4 fold greater than the deformability of the RBCs of the subject before the administering step. In some embodiments. the method maintains or reduces hemolysis of the RBC or RBCs of the subject. In some embodiments. the method maintains or increases an average blood hemoglobin concentration and/or RBC number in the subject. In some embodiments, the method increases an average blood hemoglobin concentration by at least 0.2 g/dL, by at least 0.5 g/dL, by at least 0.7 g/dL, by at least 0.8 g/dL, by at least 0.9 g/dL. or by at least 1 g/dL, relative to a baseline measurement taken in the subject before the administering step. In some embodiments, the administering step is repeated once daily, once per two days, twice per week, once per week, once per two weeks, once per three weeks, monthly, bi-monthly or yearly. In some embodiments, the effective amount administered in each dose is about 100 mg/kg to about 6.000 mg/kg. In some embodiments, the effective amount administered in each dose is about 1 milligrams per kilogram (mg/kg) to about 2.500 mg/kg. In some embodiments, the effective amount administered in each dose is from about 500 mg/kg to about 2500 mg/kg. In some embodiments, the effective amount administered in each dose is from about 1000 mg/kg to about 1500 mg/kg. In some embodiments, the effective amount administered in each dose is about 500 mg/kg, about 750 mg/kg, about 1000 mg/kg. about 1500 mg/kg, about 2000 mg/kg, or about 2500 mg/kg, optionally about 1000 mg/kg or about 1500 mg/kg. In some embodiments, the method comprises repeating the administering step once every two weeks. In some embodiments, the method comprising repeating the administering step once daily, once per two days, twice per week, once per week, once per two weeks, once per three weeks, monthly, twice per year, or yearly. In some embodiments, the subject is a human subject. In some embodiments, the RBC or RBCs have increased membrane cholesterol levels, and/or reduced deformability. In some embodiments, the subject is suffering from or at risk for Sickle Cell Disease (SCD) is Sickle Cell Anemia (SCA). In some embodiments, the cyclodextrin reduces membrane cholesterol levels, maintains or improves deformability, and/or reduces hemolysis of the RBC or RBCs. Also disclosed is a use of an effective amount of a cyclodextrin for a method described herein (for example a method according to any one of claims 1-42). Disclosed herein are cyclodextrins for use in a method of treating or preventing sickle cell disease (SCD) in a subject in need thereof, comprising administering an effective amount of the cyclodextrin. In some embodiments, the cyclodextrin for use comprises, or is, a beta (β)-cyclodextrin, optionally wherein the beta (β)-cyclodextrin comprises, or is, a hydroxypropyl-β-cyclodextrin (HPBCD), optionally wherein the hydroxypropyl-β-cyclodextrin (HPBCD) comprises, or is, a 2-hydroxypropyl-β-cyclodextrin (2-HPBCD). In some embodiments, the cyclodextrin for use reduces membrane cholesterol levels, maintains or improves deformability, and/or reduces hemolysis of the RBC or RBCs. In some embodiments, the cyclodextrin for use is for use in a method described herein (for example a method according to any one of claims 1-42). Disclosed herein are cyclodextrins for use in a method of reducing membrane cholesterol concentration, maintaining or improving deformability, and/or reducing hemolysis of a red blood cell (RBC) of a subject suffering from or at risk for sickle cell disease (SCD), comprising contacting the RBC with an effective amount of the cyclodextrin. In some embodiments, the cyclodextrin for use comprises, or is, a beta (β)-cyclodextrin, optionally wherein the beta (β)-cyclodextrin comprises, or is, a hydroxypropyl-β-cyclodextrin (HPBCD), optionally wherein the hydroxypropyl-β-cyclodextrin (HPBCD) comprises, or is, a 2-hydroxypropyl-β-cyclodextrin (2-HPBCD). In some embodiments, the cyclodextrin for use is for use in a method as described herein (for example a method according to any one of claims 1-42). In some embodiments, the method is an ex vivo, in vivo or in vitro method.

### BRIEF DESCRIPTION OF THE DRAWINGS

FIG. 1A shows effects of 5 mM 2-hydroxypropyl-β-cyclodextrin (2-HPBCD) on red blood cell (RBC) deformability parameter EImax of RBCs from healthy individuals (AA) in phosphate buffered saline (PBS).

FIG. 1B shows effects of 5 mM 2-HPBCD on RBC deformability parameter SS_{1/2} of RBCs from healthy individuals (AA) in PBS.

FIG. 2A shows effects of 5 mM 2-HPBCD on RBC deformability parameter EImax of RBCs from sickle cell disease patients (SS) in PBS.

FIG. 2B shows effects of 5 mM 2-HPBCD on RBC deformability parameter SS_{1/2} of RBCs from sickle cell disease patients (SS) in PBS.

FIG. 2C shows effects of 5 mM 2-HPBCD on RBC deformability parameter EImax of RBCs from sickle cell disease patients (SS) in plasma.

FIG. 2D shows effects of 5 mM 2-HPBCD on RBC deformability parameter SS_{1/2} of RBCs from sickle cell disease patients (SS) in plasma.

FIG. 3 shows a comparison of the Elongation Index (EImax) of RBCs from healthy individuals (AA) and sickle cell disease (SS) patients in plasma prior to administration of 2-HPBCD.

FIG. 4 shows effects of 5 mM 2-HPBCD on free hemoglobin content in supernatants after a 1 h incubation of RBC from healthy individuals (AA) in PBS.

FIG. 5A shows effects of 5 mM 2-HPBCD on free hemoglobin content in supernatants after a 1 h incubation of RBC from sickle cell disease patients (SS) in PBS.

FIG. 5B shows effects of 5 mM 2-HPBCD on free hemoglobin content in supernatants after a 1 h incubation of RBC from sickle cell disease patients (SS) with high basal free hemoglobin content in PBS.

FIG. 6A shows effects of 3 mM 2-HPBCD on free hemoglobin content in supernatants after a 1 h incubation of RBC from sickle cell disease patients (SS) in PBS.

FIG. 6B shows effects of 3 mM 2-HPBCD on free hemoglobin content in supernatants after a 1 h incubation of RBC from sickle cell disease patients (SS) in plasma.

### DETAILED DESCRIPTION

The present disclosure provides, in various embodiments, compositions and methods for treatment or prevention of sickle cell disease (SCD), such as sickle cell anemia (SCA). In some embodiments, the present disclosure provides compositions of hydroxypropyl-beta(β)-cyclodextrin (HPBCD) and uses thereof in treating or preventing SCD and/or SCA. These treatments may address an unmet need for safe and effective treatments.

Some embodiments include a method of treating or preventing SCD in a subject in need thereof. comprising administering an effective amount of a cyclodextrin to the subject. Some embodiments relate to treatment or prevention of SCD such as sickle cell anemia (SCA). For example, some embodiments include treatment or prevention of SCD or SCA by administration of a hydroxypropyl-beta-cyclodextrin such as 2-hydroxypropyl-beta-cyclodextrin (2-HPBCD).

Some embodiments include a method of reducing membrane cholesterol concentration. improving deformability, and/or reducing hemolysis of a red blood cell (RBC) of a subject suffering from or at risk for SCD, comprising contacting the RBC with an effective amount of a cyclodextrin. For example, the method may reduce membrane cholesterol concentration of the RBC. The method may improve deformability of the RBC. The method may reduce hemolysis of the RBC. The method may reduce membrane cholesterol concentration and improve deformability of the RBC. The method may reduce membrane cholesterol concentration and reduce hemolysis of the RBC. The method may improve deformability and reduce hemolysis of the RBC. The method may advantageously reduce membrane cholesterol concentration, improve deformability and reduce hemolysis of the RBC. Contacting the RBC with an effective amount of a cyclodextrin may include administering the effective amount of the cyclodextrin to the subject, where the subject comprises the RBC.

### I. Treatment Methods

Disclosed herein, in some embodiments, are methods of treating or preventing sickle cell disease (SCD) in a subject such as a human individual. Some methods include treating the SCD. Some methods include preventing the SCD. The method may include administration of a compound or composition described herein to the subject.

In some embodiments the method of treating or preventing comprises administering an effective amount of a cyclodextrin such as 2-hydroxypropyl-beta-cyclodextrin (2-HPBCD) to the subject. In some embodiments, the effective amount is an amount of cyclodextrin (e.g. HPBCD) that reduces membrane cholesterol concentration after administering of the cyclodextrin, as compared to prior to administering. In some embodiments, the effective amount is an amount of cyclodextrin (e.g. HPBCD) that maintains or improves (such as increases) red blood cell deformability in the subject after administering of the cyclodextrin, as compared to prior to administering. In some embodiments, the effective amount is an amount of cyclodextrin (e.g. HPBCD) that reduces hemolysis of a red blood cell (RBC) in the subject after administering of the cyclodextrin, as compared to prior to administering. In some embodiments the method comprises administering an amount of a cyclodextrin (e.g. HPBCD) to a subject suffering from or at risk for sickle cell discase.

In one aspect, a method of treating a disease or disorder in a human individual is provided. In some embodiments the method comprises administering an amount of a cyclodextrin such as a 2-hydroxypropyl-beta-cyclodextrin (2-HPBCD) to the subject. In some embodiments the amount of 2-hydroxypropyl-beta-cyclodextrin is an amount effective to reduce membrane cholesterol concentration after administering of the 2-HPBCD, as compared to prior to administering. In some embodiments the amount of 2-hydroxypropyl-beta-cyclodextrin is an amount effective at improving (such as increasing) red blood cell deformability in the subject after administering of the 2-HPBCD, as compared to prior to administering. In some embodiments the amount of 2-hydroxypropyl-beta-cyclodextrin is an amount effective at reducing hemolysis of a red blood cell (RBC) in the subject after administering of the 2-HPBCD, as compared to prior to administering. In some embodiments the method comprises administering an amount of 2-hydroxypropyl-beta-cyclodextrin to a subject suffering from or at risk for a sickle cell discase. In some embodiments the sickle cell disease is sickle cell anemia, sickle hemoglobin-C disease, sickle beta-plus thalassemia and/or sickle beta-zero thalassemia. In some embodiments the sickle cell disease is sickle cell anemia. In some embodiments the method comprises administering an amount of 2-hydroxypropyl-beta-cyclodextrin to a subject having sickle cell trait.

Some aspects include contacting a RBC with a cyclodextrin (such as a 2-hydroxypropyl-beta-cyclodextrin). The RBC may be contacted with the cyclodextrin in vivo. Contacting the RBC with a cyclodextrin may include administering the cyclodextrin to a subject, where the subject comprises the RBC. The RBC may be contacted with the cyclodextrin in vitro. The RBC may be contacted with the cyclodextrin ex vivo, and then added back into a subject.

In another aspect, a method of contacting a red blood cell with a cyclodextrin or composition comprising a cyclodextrin is provided, wherein the subject comprises the red blood cell. In some embodiments the method comprises contacting a red blood cell with an effective amount of 2-hydroxypropyl-beta-cyclodextrin (2-HPBCD). In some embodiments the amount of 2-hydroxypropyl-beta-cyclodextrin contacting a red blood cell is an amount effective to reduce membrane cholesterol concentration after administration of the 2-hydroxypropyl-beta-cyclodextrin, as compared to prior to administering. In some embodiments the amount of 2-hydroxypropyl-beta-cyclodextrin contacting a red blood cell is an amount effective at improving (such as increasing) red blood cell deformability in the subject after administering of the 2-hydroxypropyl-beta-cyclodextrin, as compared to prior to administering. In some embodiments the amount of 2-hydroxypropyl-beta-cyclodextrin contacting a red blood cell is an amount effective at reducing hemolysis of a red blood cell in the subject after administering of the 2-hydroxypropyl-beta-cyclodextrin, as compared to prior to administering. In some embodiments the method comprises administering an amount of 2-hydroxypropyl-beta-cyclodextrin contacting a red blood cell to a subject suffering from or at risk for sickle cell disease.

Some embodiments include an administering step, such as a step of administering a cyclodextrin to a subject. In some embodiments, the administering is by intravenous administration. In some embodiments, the administering is by parenteral administration, including intravenous, intravascular, intramuscular, subcutaneous, intrathecal, depot, or peristaltic pump administration. In some embodiments, the administering of the composition described herein comprises an intravenous administration. The administration may include an injection. The administration may be repeated. In some embodiments, the administering step is repeated once daily, once per two days, twice per week, once per week, once per two weeks, once per three weeks, monthly, bi-monthly, twice per year, or yearly.

In some embodiments, before the administering step (e.g. before the first administering step), a RBC of the subject exhibits increased membrane cholesterol levels compared to a healthy subject. In some embodiments, before the administering step (e.g. before the first administering step), a RBC of the subject exhibits reduced deformability compared to a healthy subject.

In some embodiments, after the administering step (e.g., after the first administering step, or after a subsequent administering step), membrane cholesterol levels of red blood cells (RBCs) of the subject are at least 10%, at least 20%, at least 30%, at least 40%, or at least 50% lower than than the membrane cholesterol levels of RBCs of the subject before the administering step (e.g., before the first administering step). In some embodiments, membrane cholesterol levels of RBCs of the subject is between about 10% to about 20%, between about 20% to about 30%, between about 30% to about 40%, or between about 40% to about 50% lower than the membrane cholesterol levels of RBCs of the subject before the administering step (e.g., before the first administering step).

In some embodiments, the method reduces membrane cholesterol levels in a RBC of the subject. In some embodiments, the method reduces membrane cholesterol in a RBC of the subject by administering an amount of 2-hydroxypropyl-beta-cyclodextrin (2-HPBCD) to the subject. In some embodiments the sickle cell disease is sickle cell anemia.

In some embodiments, the method maintains deformability or increases deformability of RBC of the subject. In some embodiments, the method increases RBC deformability in the subject by at least about 5%, at least about 10%, or at least about 15%, by at least about 10% to about 20%, by at least about 10% to about 30%, by at least about 10% to about 40%, by at least about 10% to about 50%, by at least about 10% to about 60%, by at least about 10% to about 70%, by at least about 10% to about 80%, by at least about 10% to about 90% or by at least about 10% to about 100% of the deformability of RBCs of a healthy subject.

In some embodiments, after the administering step (e.g., after the first administering step, or after a subsequent administering step), the RBC deformability in the subject is at least about 5%, at least about 10%, or at least about 15%, at least about 10% to about 20%, at least about 10% to about 30%, at least about 10% to about 40%, at least about 10% to about 50%, at least about 10% to about 60%, at least about 10% to about 70%, at least about 10% to about 80%, at least about 10% to about 90% or at least about 10% to about 100% greater than the deformability of the RBCs of the subject before the administering step (e.g., before the first administering step).

In some embodiments, the RBC deformability is assessed by the elongation index at infinite shear stress (EImax) and/or by the shear stress required to reach half of this maximum elongation index (SS_{1/2}). For example, an increase in RBC deformability in the subject by at least about 5% may be determined by an improvement (such as an increase) in the EImax by at least about 5% relative to a control or baseline measurement (such as a control or baseline measurement taken in the subject prior to administration of the cyclodextrin). Additionally, or alternatively, an increase in RBC deformability in the subject by at least about 5% may be determined by an improvement (such as a decrease) in the shear stress required to reach half of this maximum elongation index (SS1/2) by at least about 5% relative to a control or baseline measurement (such as a control or baseline measurement taken in the subject prior to administration of the cyclodextrin). In some embodiments, an increase in RBC deformability in the subject is determined by an improvement in both EImax and SS_{1/2}.

In some embodiments, after the administering step (e.g., after the first administering step, or after a subsequent administering step), the deformability of a RBC of the subject is at least about 1.1fold, about 1.2 fold, about 1.3 fold, about 1.4 fold, about 1.5 fold, about 2 fold, about 3 fold, or about 4 fold greater than the deformability of the RBC of the subject before the administering step (e.g., before the first administering step). In some embodiments, after the administering step (e.g., after the first administering step, or after a subsequent administering step), the deformability of a RBC of the subject is between about 1.1 fold to about 1.2 fold, about 1.2 fold to about 1.3 fold, about 1.3 fold to about 1.4 fold, about 1.4 fold to about 1.5 fold, about 1.5 fold to about 2 fold, about 2 fold to about 3 fold or about 3 fold to about 4 fold greater than the deformability of the RBCs of the subject before the administering step (e.g., before the first administering step).

In some embodiments, the method maintains or reduces hemolysis of the RBC or RBCs of the subject. In some embodiments, the method maintains or reduces hemolysis of the RBC or RBCs of the subject by administering an amount of 2-hydroxypropyl-beta-cyclodextrin (2-HPBCD) to the subject. In some embodiments the sickle cell disease is sickle cell anemia.

The reduction in hemolysis may result in an increase in RBC number and/or blood hemoglobin concentration (such as blood hemoglobin concentration) which can be clinically beneficial for subjects suffering from or at risk for SCD. In some embodiments, the method maintains or increases an average blood hemoglobin concentration measurement in the subject, relative to a baseline measurement. In some embodiments, the method increases an average blood hemoglobin concentration by at least 0.2 g/dL, by at least 0.5 g/dL, by at least 0.7 g/dL, by at least 0.8 g/dL, by at least 0.9 g/dL, or by at least 1 g/dL, relative to a baseline measurement (such as a baseline measurement taken in the subject prior to administration of the cyclodextrin). Preferably, the method increases an average blood hemoglobin concentration by at least 1 g/dL, relative to a baseline measurement (such as a baseline measurement taken in the subject prior to administration of the cyclodextrin).

In some embodiments, the method increases the average blood hemoglobin measurement in the subject by at least about 5%, by at least about 5% to about 10%, by at least about 10% to about 20%. by at least about 20% to about 30%, by at least about 30% to about 40%, by at least about 40% to about 50%, by at least about 50% to about 60%, by at least about 60% to about 70%, by at least about 70% to about 80%, by at least about 80% to about 90% or by at least about 90% to about 100%, in the subject relative to a baseline average blood hemoglobin concentration measurement before the administering step (e.g., before the first administering step).

In some embodiments, the hemolysis of the RBC or RBCs is assessed by a plasma free hemoglobin release measurement as described hereinbelow. In some embodiments, the reduced hemolysis may be determined by a decrease in the plasma free hemoglobin release measurement. In some embodiments, the free hemoglobin release in plasma or in incubation medium measurement decreases by at least 5% or 10% relative to a control or baseline measurement (such as a control or baseline measurement taken in the subject prior to administration of the cyclodextrin).

Disclosed herein, in some embodiments, are methods of reducing membrane cholesterol concentration of a red blood cell (RBC) of a subject suffering from or at risk for sickle cell disease (SCD) comprising contacting the RBC with an effective amount of a cyclodextrin. In some embodiments, the methods reduce excess membrane cholesterol concentration. Disclosed herein, in some embodiments, are methods of treating or preventing sickle cell disease (SCD) in a subject in need thereof comprising administering an effective amount of a cyclodextrin. In some embodiments, the membrane cholesterol levels in red blood cells (RBCs) of the subject are at least 10%, 20%, 30%, 40%, 50%, 60%, 70%, 80%, 90%, 100%, 150%, 200%, 250%, 300%, 400%, 500%, or more, higher than membrane cholesterol levels in red blood cells (RBCs) of a healthy subject. In some embodiments, the cyclodextrin is a β-cyclodextrin. In some embodiments, the β-cyclodextrin is a hydroxypropyl-β-cyclodextrin. In some embodiments, the hydroxypropyl-β-cyclodextrin is a 2-hydroxypropyl-β-cyclodextrin. In some embodiments, the effective amount is about 1 milligrams per kilogram (mg/kg) to about 2.500 mg/kg. In some embodiments, the administering step is repeated once daily, once per two days, twice per week, once per week, once per two weeks, or once per three weeks. In some embodiments, the RBC or RBCs have increased membrane cholesterol levels, and/or reduced deformability. In some embodiments, the SCD is sickle cell anemia (SCA). The subject may be suffering from or at risk of SCA. In some embodiments, the cyclodextrin reduces membrane cholesterol levels, maintains or improves deformability, and/or reduces hemolysis of the RBC or RBCs. Disclosed herein is the use of an effective amount of a cyclodextrin for a method, substantially as described herein.

Some methods include reducing membrane cholesterol concentration of a red blood cell (RBC) by contacting the RBC with a cyclodextrin. Some methods include increasing deformability of a RBC by contacting the RBC with a cyclodextrin. Some methods include reducing hemolysis of a RBC by contacting the RBC with a cyclodextrin. The contact or method may be performed in a subject having a SCD.

### II. Subjects

Disclosed herein, in some embodiments, are methods that include treatment of a subject. For example, the subject may be administered a compound or composition described herein, such as a cyclodextrin or a composition comprising a cyclodextrin.

In some embodiments, the subject is in need of treatment for sickle cell disease (SCD). In some embodiments, the subject has sickle cell anemia (SCA), sickle hemoglobin-C disease, sickle beta-plus thalassemia and/or sickle beta-zero thalassemia. The subject may have SCD. The subject may be identified as having SCD or SCA. For example, the subject may have been diagnosed as having SCD. Some embodiments include selecting a subject who has SCD such as SCA. The subject may be at risk of having SCD. In some embodiments, the subject is at risk of having SCA. The subject may be identified as at risk of having SCD or SCA. Some embodiments include selecting a subject at risk of having SCD such as SCA. The treatment may be prophylactic. In some embodiments, the subject exhibits a symptom or symptoms of SCD. In some embodiments, the subject exhibits a symptom or symptoms of sickle hemoglobin-C disease, sickle beta-plus thalassemia and/or sickle beta-zero thalassemia. In some embodiments. RBC deformability in the subject is associated with SCA complications such as hemolytic anemia and/or vaso-occlusive crisis.

SCD may include an inherited red blood cell disorder that affects hemoglobin. In healthy patients without SCD, red blood cells may be disc-shaped and flexible enough to move easily through the blood vessels. However. SCD red blood cells may be crescent-or sickle-shaped, or may not bend or move easily, and can block blood flow to the rest of the body. The blocked blood flow through the body can lead to further problems. The subject may present with any of these problems, or the treatment may help to address (e.g. improve or prevent) any of these effects. In some embodiments, the subject is homozygous for a genetic mutation that results in SCD.

In some embodiments, the SCD includes SCA. In some embodiments, the SCD is SCA. SCA may include a severe hereditary form of anemia in which a mutated form of hemoglobin distorts the red blood cells into a crescent or sickle shape in SCA. Sickle shaped red blood cells may break apart easily and die. Red blood cells may usually live for about 120 days before they need to be replaced, but sickle cells may die in 10-20 days leading to a shortage of red blood cells (anemia). Without enough red blood cells, the body is unable to get enough oxygen and this may cause fatigue and end organ damage (without exceptions). The subject may present with any of these problems, or the treatment may help to address (e.g. improve or prevent) any of these effects.

The subject may have sickle cell trait. Sickle cell trait is a condition in which the subject has an abnormal allele of a hemoglobin beta gene (i.e. the subject is heterozygous for HbS). A sickle cell trait subject may produce normal and abnormal hemoglobin. The normal hemoglobin may compensate for the abnormal hemoglobin that is produced, and so the subject may have fewer complications associated with the abnormal hemoglobin than a person with a homozygous allele causing the SCD.

The subject may be an animal. The subject may be a vertebrate. The subject may be a mammal. The subject may be a primate. The subject may be human. The subject may be a patient such as an SCD patient. The subject may be non-human. Examples of non-human subjects may include a non-human animal such as a non-human primate, rabbit, hamster, gerbil, pig, cow, sheep, goat, guinea pig, rat, mouse, squirrel, wolf, fox, horse, zebra, giraffe, hyena, elephant, cat, dog, llama, or ferret.

### III. Cyclodextrins

Disclosed herein, in some embodiments, are cyclodextrins (e.g. HPBCD). The cyclodextrin (e.g. HPBCD) may be used in a method of treatment such as treatment of sickle cell disease (SCD). For the avoidance of doubt, any and all disclosures of methods of treatment provided herein should also be read as disclosing the cyclodextrin (e.g. HPBCD) or a composition or formulation comprising the same, for use in the described methods of treatment. The cyclodextrin may be administered to a subject, or may be used in a method herein. The cyclodextrin may be included in a composition such as a pharmaceutical composition.

In some embodiments, the cyclodextrin comprises, or consists of, beta (β)-cyclodextrin. In some embodiments, the-cyclodextrin comprises, or consists of, hydroxypropyl-β-cyclodextrin (HPBCD). In some embodiments, the HPBCD comprises, or consists of, 2-hydroxypropyl-β-cyclodextrin (2-HPBCD).

In some embodiments, the β-cyclodextrin comprises, or consists of, a mixture of 2-hydroxypropyl-β-cyclodextrin (2-HPBCD) molecules. In some embodiments, the mixture comprises at least 10% 2-hydroxypropyl-β-cyclodextrin (2-HPBCD) molecules selected from beta-cyclodextrin substituted with four hydroxypropyl groups (“DS-4”), beta-cyclodextrin substituted with five hydroxypropyl groups (“DS-5”), beta-cyclodextrin substituted with six hydroxypropyl groups (“DS-6”), beta-cyclodextrin substituted with seven hydroxypropyl groups (“DS-7”), and beta-cyclodextrin substituted with eight hydroxypropyl groups (“DS-8”). For example, the mixture may include 10%-20%, 10%-30%, 10%-40%, 10%-50%, 10%-60%, 10%-70%, or 10%-80%, or any other range of 2 of the aforementioned percentages, 2-HPBCD molecules substituted with 4. 5. 6. 7, and/or 8 hydroxypropyl groups. Any of the percentages here may be a weight % (e.g. wt/wt or wt/vol) or a molar %. In some embodiments, the mixture comprises less than 1% beta-cyclodextrin substituted with one hydroxypropyl group (“DS-1”) and less than 1% unsubstituted beta-cyclodextrin (“DS-0”). In some embodiments, the mixture comprises no beta-cyclodextrin substituted with one hydroxypropyl group (“DS-1”) and/or no unsubstituted beta-cyclodextrin (“DS-0”). In some embodiments, the mixture comprises at least 10% beta-cyclodextrin substituted with four hydroxypropyl groups (“DS-4”). For example, the mixture may include 10%-20%, 10%-30%, 10%-40%, 10%-50%, 10%-60%, 10%-70%, or 10%-80%, or any other range of 2 of the aforementioned percentages, of DS-4. In some embodiments, the mixture comprises at least 10% beta-cyclodextrin substituted with five hydroxypropyl groups (“DS-5”). For example, the mixture may include 10%-20%. 10%-30%. 10%-40%, 10%-50%, 10%-60%, 10%-70%, or 10%-80%, or any other range of 2 of the aforementioned percentages, of DS-5. In some embodiments, the mixture comprises at least 10% beta-cyclodextrin substituted with six hydroxypropyl groups (“DS-6”). For example, the mixture may include 10%-20%, 10%-30%, 10%-40%, 10%-50%, 10%-60%, 10%-70%, or 10%-80%, or any other range of 2 of the aforementioned percentages, of DS-6. In some embodiments, the mixture comprises at least 10% beta-cyclodextrin substituted with seven hydroxypropyl groups (“DS-7”). For example, the mixture may include 10%-20%, 10%-30%, 10%-40%, 10%-50%, 10%-60%, 10%-70%, or 10%-80%, or any other range of 2 of the aforementioned percentages, of DS-7. In some embodiments, the mixture comprises at least 10% beta-cyclodextrin substituted with eight hydroxypropyl groups (“DS-8”). For example, the mixture may include 10%-20%, 10%-30%, 10%-40%, 10%-50%, 10%-60%, 10%-70%, or 10%-80%, or any other range of 2 of the aforementioned percentages, of DS-8.

In some embodiments, the 2-hydroxypropyl-β-cyclodextrin (2-HPBCD) molecules in the mixture have an average degree of substitution of from about 3 to about 10. For example, the average degree of substitution may be 3, 4, 5, 6, 7, 8, 9, or 10, or a range defined by any two of the aforementioned integers.

In some embodiments, the cyclodextrin is part of a plurality or mixture of distinct cyclodextrins. The plurality of distinct cyclodextrins may be included as part of a mixture of cyclodextrins. The plurality of distinct cyclodextrins may be or include a plurality of distinct HPBCDs. In some embodiments, the plurality of cyclodextrins, optionally a plurality of distinct HPBCDs, have an average molecular mass of from about 1350 Da to about 1750 Da. In some embodiments, the plurality of HPBCDs has an average molecular mass of from about 1350 Da to about 1450 Da, from about 1450 Da to about 1550 Da, from about 1550 Da to about 1650 Da, or about 1650 Da to about 1750 Da. In some embodiments, the plurality of cyclodextrins, optionally a plurality of distinct HPBCDs, has an average degree of substitution of from about 4 to about 10. For example, in some embodiments, the average degree of substitution is about 4, about 5, about 6, about 7, about 8, about 9, or about 10.

A 2-hydroxypropyl-beta-cyclodextrin may include a beta-cyclodextrin wherein one or more hydroxyl of the beta-cyclodextrin is substituted with a 2-hydroxypropyl. In some embodiments, a 2-hydroxypropyl-beta-cyclodextrin also includes a beta-cyclodextrin wherein one or more hydroxyl of the beta-cyclodextrin is substituted with an oligomerized or polymerized hydroxypropyl.

### IV. Compositions

Disclosed herein, in some embodiments, are compositions. The composition may include a cyclodextrin, such as HPBCD or 2-HPBCD. The composition may include a plurality of cyclodextrins. The composition may be a pharmaceutical composition. The composition may be administered to a subject, or may be used in a method herein.

The composition may include a carrier. Some examples of carriers may include water, a buffer, or a saline solution. The carrier may include a pharmaceutically acceptable carrier.

### V. Definitions

It is to be understood that the embodiments herein are provided by way of example only, and that various alternatives to the embodiments of the disclosure described herein may be employed in practicing the invention. Numerous variations, changes, and substitutions will now occur to those skilled in the art without departing from the invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole.

The singular forms “a,” “an,” and “the” are intended to include the plural forms as well, unless the context clearly indicates otherwise.

As used herein, the term “about” means a range of values including the specified value, which a person of ordinary skill in the art would consider reasonably similar to the specified value. For example, about means within a standard deviation using measurements generally acceptable in the art. For example, about means a range extending to +/−10%, +/−5%, +/−3%, or +/−1% of the specified value.

Some embodiments include administering a compound or a composition. Administering may include providing a composition to a subject in a manner that permits the composition to have its intended effect. Administration for sickle cell disease (SCD) may be performed by intravenous injection or any other suitable route.

Treating or treatment may include any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, diminishment of the reoccurrence of disease, and remission, whether partial or total and whether detectable or undetectable. A treatment may include any cure, amelioration, or prevention of a disease. Treatment may prevent the disease from occurring: inhibit the disease's spread: relieve the disease's symptoms: fully or partially remove the disease's underlying cause: shorten a disease's duration: or do a combination of these things. Methods of treatment as disclosed herein may include use of the compounds, pharmaceutical compositions, or pharmaceutical formulations provided herein for the treatment of any indication described herein, and may include the compounds, pharmaceutical compositions, or pharmaceutical formulations provided herein for the use in treating any indication described herein.

Some embodiments include administration of an effective amount. The effective amount may improve a parameter. In some embodiments, for the given parameter, an effective amount will show an increase or decrease of at least 5%, 10%, 15%, 20%, 25%, 40%, 50%, 60%, 75%, 80%, 90%, or at least 100%. Efficacy can also be expressed as-fold increase or decrease. For example, an effective amount can have at least a 1.2-fold, 1.5-fold, 2-fold, 5-fold, or more effect over a control. An effective amount may be or include a sufficient amount of a compound, pharmaceutical composition, or pharmaceutical formulation being administered which relieves, to some extent, one or more of the symptoms of the disease or condition being treated, or reduces the underlying cause of the disease or condition being treated. In some embodiments, the result is a reduction or alleviation of a sign, symptom, or cause of a disease, or any other desired alteration of a biological system. For example, an effective amount for therapeutic use may be an amount of a compound, pharmaceutical composition, or pharmaceutical formulation as disclosed herein required to provide a clinically significant decrease in disease symptoms or underlying cause of the disease (e.g., without undue adverse side effects). In some embodiments, an effective amount is or includes a pharmaceutically effective amount of a compound, pharmaceutical composition, or pharmaceutical formulation as disclosed herein required to provide a pharmaceutical effect to prevent, reduce, ameliorate, or inhibit progression of at least one symptom of a disease, disorder or condition in the subject in need of the administration of such amount of the composition. In some embodiments, an effective amount in any individual case is determined using techniques, such as a dose escalation study. In some embodiments, an effective amount of a compound, pharmaceutical composition, or pharmaceutical formulation may be an amount effective to achieve a desired effect or therapeutic improvement (e.g., without undue adverse side effects). An effective amount of a compound, pharmaceutical composition, or pharmaceutical formulation disclosed herein may be an amount effective to achieve one or more desired outcomes (e.g., alleviate a symptom of SCD). It should be understood that, in some embodiments. “an effective amount” varies from subject to subject, due to variation in metabolism of the composition, age, weight, general condition of the subject, concomitant medications the subject may be taking, the condition being treated, the severity of the condition being treated, and the judgment of the prescribing physician.

In some instances, the disease or condition being treated is sickle cell disease (SCD). The SCD may include, or may be, sickle cell anemia (SCA), sickle hemoglobin-C disease, sickle beta-plus thalassemia, sickle beta-zero thalassemia or a consequential disease of any of the aforementioned examples. SCD may include a group of red blood cell disorders that affect hemoglobin, a protein that carries oxygen through the body. SCD may be inherited, for example through a mutation in a hemoglobin beta gene. SCD may include sickle cell anemia. Sickle cell anemia may appear when a person is homozygous for an abnormal allele of a hemoglobin beta gene. A person may in some instances carry an abnormal allele of a hemoglobin beta gene without being anemic. Sickle cell trait is a condition in which a person is heterozygous for an abnormal allele of a hemoglobin beta gene.

Prevention or preventing may include inhibiting the onset of a disease in a subject or patient which may be at risk and/or predisposed to the disease but does not yet experience or display any or all of the pathology or symptomatology of the disease, and/or slowing the onset of the pathology or symptomatology of a disease in a subject or patient which may be at risk and/or predisposed to the disease but does not yet experience or display any or all of the pathology or symptomatology of the disease. The prevention may be complete (e.g. resulting in no detectable symptoms) or partial, such that fewer symptoms are observed than would likely occur absent treatment.

### NUMBERED EMBODIMENTS

Some embodiments include any of the following:
1. A method of reducing membrane cholesterol concentration of a red blood cell (RBC) of a subject suffering from or at risk for Sickle Cell Disease (SCD), comprising contacting the RBC with an effective amount of a cyclodextrin.
2. A method of treating or preventing Sickle Cell Disease (SCD) in a subject in need thereof, comprising administering an effective amount of a cyclodextrin.
3. The method of embodiment 2, wherein the membrane cholesterol levels in red blood cells (RBCs) of the subject is at least 10%, 20%, 30%, 40%, 50%, 60%, 70%, 80%, 90%, 100%, 150%, 200%, 250%, 300%, 400%, 500%, or more, higher than the membrane cholesterol levels in red blood cells (RBCs) of a healthy subject.
4. The method of any one of embodiments 1 to 3, wherein the cyclodextrin is a beta(β)-cyclodextrin.
5. The method of embodiment 4, wherein the β-cyclodextrin is a hydroxypropyl-β-cyclodextrin (HPBCD).
6. The method of of embodiment 5, wherein the HPBCD is a 2-hydroxypropyl-β-cyclodextrin (2-HPBCD).
7. The method of any one of embodiments 2 to 6, wherein the effective amount is about 1 milligrams per kilogram (mg/kg) to about 2,500 mg/kg.
8. The method of any one of embodiments 2 to 7, wherein the administering step is repeated once daily, once per two days, twice per week, once per week, once per two weeks, or once per three weeks.
9. The method of any one of embodiments 1 to 8, wherein the RBC or RBCs have increased membrane cholesterol levels, and/or reduced deformability.
10. The method of any one of embodiments 2 to 8, wherein the subject is suffering from or at risk for Sickle Cell Disease (SCD) is Sickle Cell Anemia (SCA).
11. The method of any one of embodiments 1 to 9, wherein the cyclodextrin reduces membrane cholesterol levels, maintains or improves deformability, and/or reduces hemolysis of the RBC or RBCs.
12. Use of an effective amount of a cyclodextrin for a method, substantially as described herein.

### EXAMPLES

The following Examples are merely illustrative and are not intended to limit the scope or content of the invention in any way.

### Example 1.

In vitro studies measure the effect of HPBCD treatment on RBC membrane cholesterol content and deformability in RBC from 25 healthy volunteers (AA) and 10 homozygotes sickle cell disease (SS) patients. In the SS patients the effect of HPBCD on the sickling process will be further investigated.

Correlations between RBC membrane cholesterol concentration, RBC deformability and SS RBC sickling are tested and confirmed.

Additional patients are recruited to reach statistical significance and/or to compare functions of reference and competitor compounds such as methyl-cyclodextrin.

### Step 0: Assay Dose Range and Incubation Conditions

Incubate RBC from Healthy Volunteers in saline or PBS and compare with 20% homologous plasma.

Final HPBCD concentrations: 0.1, 0.3, 1, 3 and 10 mM for 2, 6 and 12 h.

Initial hemolysis conditions will be determined at 5% hematocrite and 0 or 20% plasma/saline.

### Step 1

Incubate washed RBC from the 3 groups AA and SS with HPBCD select concentrations, conditions and final concentration from step 0.

Measure hemolysis.

Measure RBC membrane cholesterol and phospholipids.

Measure cholesterol in HPBCD fraction post incubation.

Measure major plasma apolipoproteins (ApoA1 and ApoB).

### Step 2

Incubate washed RBC from the 3 groups AA and SS with HPBCD select concentrations, conditions and final concentration from step 0.

Measure RBC deformability and sickling by ektacytometry (shear stress gradient and oxygen gradient).

Measure RBC membrane cholesterol and phospholipids (if needed). Measure cholesterol in HPBCD fraction post incubation.

### Step 3

Incubate washed RBC from AA and SS with HPBCD select concentrations, conditions and final concentration from step 0.

Measure RBC aggregation.

Measure Phosphatidylserine externalization.

Measure deformability and sickling with Lorrca® Mechatronic equipment.

### Example 2. Effects of 2-HPBCD on Red Blood Cells from Healthy Subjects and Sickle Cell Anemia Patients: Impact on RBC Deformability and Hemolysis

As provided below, the deformability of red blood cells (RBC) from healthy volunteers and sickle cell disease (SCD) patients was investigated.

It was confirmed that the RBCs were less deformable (as measured by the maximum elongation index at infinite shear stress (EImax) by ektacytometry) in SCD subjects (SS) than healthy volunteers (AA). The RBC deformability and free hemoglobin release after incubation with 2-hydroxypropyl-β-cyclodextrin (2-HPBCD) was measured.

Surprisingly, incubation with 2-HPBCD (3 mM or 5 mM) increased deformability of SCD RBCs. Also surprisingly, 2-HPBCD decreased free hemoglobin released following one hour incubation of RBCs from SCD patients. This observation will have an impact on RBC fragility and hemolysis. By decreasing hemolysis, 2-HPBCD may diminish the toxicity of free hemoglobin released by fragile RBCs. Removing excess cholesterol from RBC may be achieved using 2-HPBCD.

### Aim

A goal of the experiments in this example was to assess the effect of treatment using (2-hydroxypropyl)-β-cyclodextrin (2-HPBCD) on RBC deformability and hemolysis in RBC from healthy volunteers (AA) and homozygous sickle cell anemia (SS) patients.

### Materials and Methods

Subjects and blood preparation: 17 sickle cell patients (24.1 +/-12.1 years old) and 6 healthy individuals (26 +/−3.9 years old) were included in this study. 12 sickle cell patients were at steady state at the time of inclusion and 5 had vaso-occlusive crisis the day of inclusion. 14 patients were under hydroxyurea therapy. Blood was drawn into EDTA tubes, centrifuged, and plasma and buffy coat were removed and kept until utilization. RBCs were washed in PBS 1X (Phosphate Buffered Saline, 1X Solution) and resuspended at hematocrit 20% either in PBS 1X or in autologous plasma. Then RBCs were incubated for 1 h at 37° C, with 3 or 5 mM 2-HPBCD or vehicle for control condition (Ctrl). The vehicle for control condition was the PBS IX or plasma respectively, i.e., without addition of the 2-HPBCD.

Red blood cell deformability: RBC deformability was assessed at 37° C, at shear stresses ranging from 0.3 to 30 Pa by laser diffraction analysis (ektacytometry), using a Laser-assisted Optical Rotational Cell Analyzer (LORRCA MaxSis, RR Mechatronics. Hoorn. The Netherlands). 10 μL of RBC suspensions were mixed with 1 ml polyvinylpyrrolidone (PVP: viscosit≈30 cP) and sheared into a Couette system. A laser beam was directed through the samples. The shear stress-induced deformation of RBCs affected the laser beam's diffraction pattern, which was measured by the LORRCA software, and used to calculate an elongation index. A higher elongation index represents greater RBC deformability. The Lineweaver-Burke method was used to calculate the maximum elongation index at infinite shear stress (EImax) and the shear stress required to reach half of this maximum elongation index (SS_{1/2}).

Free hemoglobin assay: following incubations. RBC suspensions were centrifuged, and supernatant was collected and stored at 80° C, until analysis. Free hemoglobin in the supernatants was then quantified according to Drabkin's reagent hemoglobin assay. This procedure was based on oxidation of hemoglobin and its derivatives to methemoglobin in the presence of alkaline potassium ferricyanide. Methemoglobin reacts with potassium cyanide to form cyanmethemoglobin, which has maximum absorption at 540 nm. The color intensity determined at 540 nm with a spectrophotometer was proportional to the total hemoglobin concentration. A standard curve with known concentrations of hemoglobin was also prepared to allow for free hemoglobin quantification in the supernatants.

Statistical analysis: effects of treatments on deformability parameters (EImax and SS_{1/2}) and free hemoglobin concentration were analyzed by using a paired t-test or Wilcoxon test when appropriate. Comparisons of RBC deformability between healthy individuals and patients were achieved using Student's t-test. A p value<0.05 was considered as significant. Analyses were performed with Graphpad Prism 9 software.

### Red Blood Cell Deformability

It was demonstrated that 2-hydroxypropyl-β-cyclodextrin (2-HPBCD) increased red blood cell (RBC) deformability, as tested using RBCs from 17 sickle cell anemia patients (SS) and 6 healthy individuals (AA).

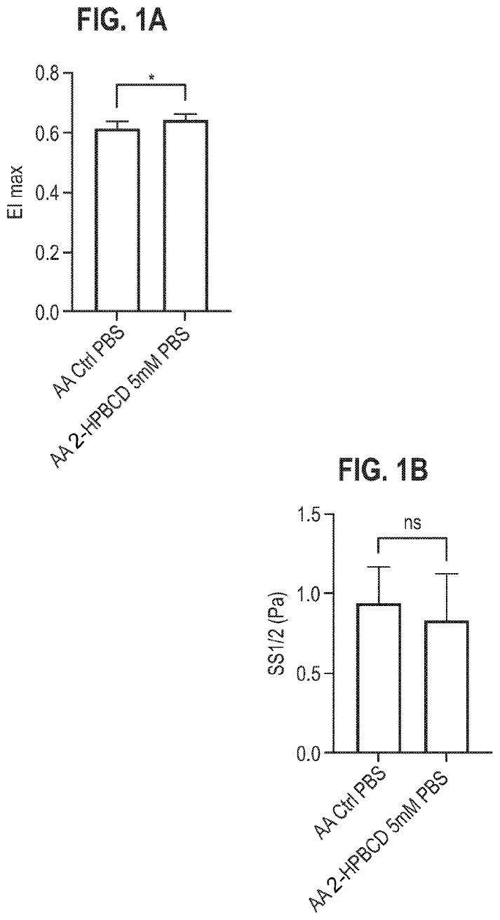

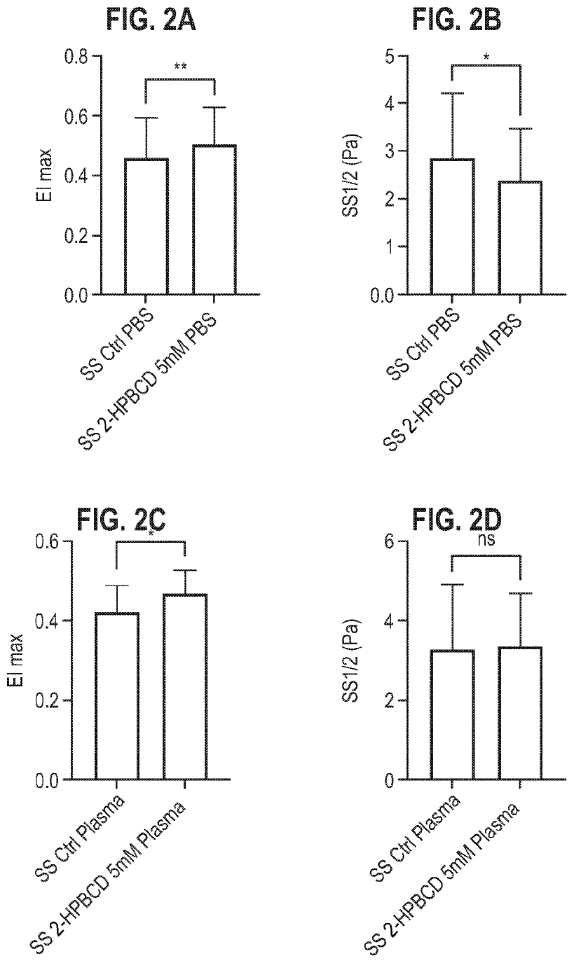

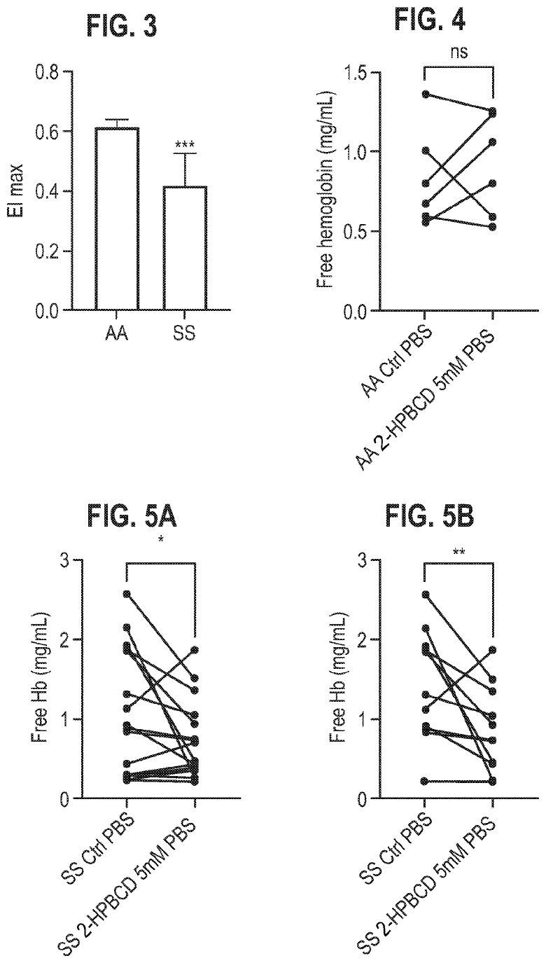

5 mM 2-HPBCD significantly increased RBC EImax from healthy controls (p<0.05. +4.4%) (FIG. 1A). 5 mM 2-HPBCD had no significant effect on SSin on RBCs from healthy controls (FIG. 1B). 2-HPBCD treatment increased sickle cell RBCs EImax (p<0.01. +12.7%) when incubated in PBS (FIG. 2A), confirming that deformability of the RBC was improved. 2-HPBCD treatment decreased sickle cell RBC SS_{1/2} (p<0.5, −8.1%) when incubated in PBS (FIG. 2B), confirming that the rigidity of the RBC decreased and hence that deformability of the RBC was improved. 2-HPBCD treatment improved sickle cell RBC Eimax (p<0.01. +12.4%) when incubated in plasma (FIG. 2C). 2-HPBCD treatment did not significantly change SS_{1/2} in sickle cells RBCs when incubated in plasma (FIG. 2D). Comparison of Eimax of RBCs from healthy individuals and sickle cell patients showed decreased EImax in RBCs from sickle cell patients (p<0.001 vs AA) (FIG. 3).

These data highlight the usefulness of treatments that include administering 2-HPBCD to a subject who has SCD, or contacting a sickle cell with 2-HPBCD. For example, the 2-HPBCD treatment may be useful for improving a RBC deformability measurement such as an EImax measurement or an SS_{1/2} measurement. In some cases, it may be useful to treat a healthy subject, or a subject without SCD, with 2-HPBCD to improve a deformability measurement such as an EImax measurement or an SS_{1/2} measurement. The improvement may be by at least 5% or 10% relative to a control or baseline measurement, such as a baseline measurement taken in the subject prior to administering the 2-HPBCD.

### Hemolysis

This example demonstrated that 2-HPBCD decreased RBC hemolysis, as tested on RBCs from 17 sickle cell anemia patients (SS). The 2-HPBCD did not decrease RBC hemolysis from 6 healthy individuals (AA).

5 mM 2-HPBCD had no significant effect on free hemoglobin release in the supernatant during incubation of RBCs from healthy control individuals (FIG. 4). 5 mM 2-HPBCD significantly decreased free hemoglobin released by RBCs from sickle cell anemia patients (p<0.05, −6.4%) (FIG. 5A). The decrease in free hemoglobin was more pronounced when patients with low basal (Crtl) free hemoglobin were removed from the statistical analysis (p<0.01, −29.9%) (FIG. 5B).

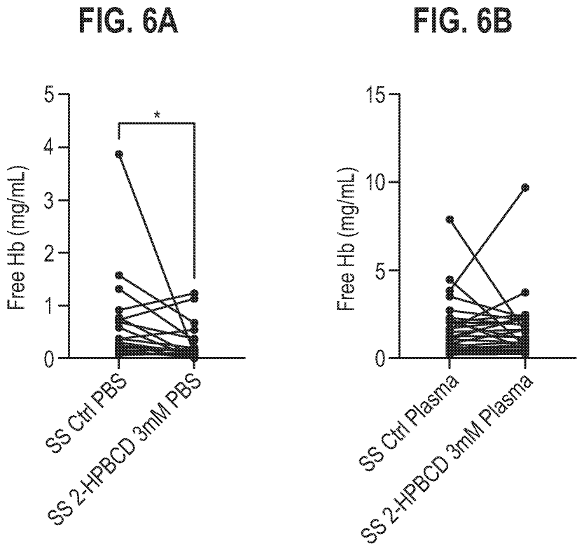

3 mM 2-HPBCD significantly decreased free hemoglobin released by RBCs from sickle cell disease patients (p<0.05) in PBS (FIG. 6A).

These data highlight the usefulness of treatments that include administering 2-HPBCD to a subject who has SCD, or contacting a sickle cell with a cyclodextrin. For example, the 2-HPBCD treatment may be useful for improving a hemolysis measurement such as a free hemoglobin release measurement. In some cases, it may be useful to treat a healthy subject, or a subject without SCD, with 2-HPBCD to improve a hemolysis measurement such as a free hemoglobin release measurement. The improvement may be by at least 5% or 10% relative to a control or baseline measurement, such as a baseline measurement taken in the subject prior to administering the 2-HPBCD.

### Study Conclusions

Example 2 shows that in vitro treatment of RBCs from SCA patients with 2-HPBCD increased the RBC deformability when incubated in both PBS and plasma. As decreased RBC deformability is a major factor in SCA pathophysiology, these results are of clinical interest. RBC deformability may also be associated with various SCA complications such as hemolytic anemia or vaso-occlusive crisis. The elongation index may be used as a prognostic rheological biomarker marker of future complication in high risk SCD patients.

Surprisingly, 2-HPBCD was also effective in reducing the release of free hemoglobin content in the supernatant of the incubated SCA RBCs, which further indicates that 2-HPBCD is useful to decrease RBC fragility and susceptibility to lysis.

It was confirmed that RBCs from SCA may be less deformable than RBCs of healthy volunteers without SCA. Cholesterol may play a role in RBC deformability. Increased membrane cholesterol content may be a characteristic of RBCs from some SCA patients, and this can adversely affect RBC deformability. Therefore, the inventors believe that, by removing membrane cholesterol content, 2-HPBCD improves RBC membrane elasticity and deformability, thus preventing cell rupture under stressful conditions in the case of a SCD such as SCA.

One of the unexpected findings of this investigation was the observation of a decreased free hemoglobin release in the incubation medium in which RBCs from SCD patients were incubated with 2-HPBCD. RBCs from SCD patients may be fragile and release free hemoglobin when subjected to various stressors.

### Example 3

In vitro studies to measure the effect of 2-hydroxypropyl-β-cyclodextrin (2-HPBCD) treatment on hemolysis and deformability in RBCs from healthy volunteers and homozygous sickle cell disease patients will be performed. Samples from each patient will be treated with one of the following: 2-HPBCD, methyl-cyclodextrin (MCD), apolipoprotein A-1 (ApoA1), voxelotor or unsubstituted cyclodextrin: and then hemolysis and RBC deformability assays will be performed. The results of this experiment are expected to show a combination of both reduced hemolysis and increased RBC deformability with 2-HPBCD treatment, and not in the other treatment groups.

### Example 4

In vitro studies to measure the effect of 2-HPBCD treatment on hemolysis and deformability in RBCs from healthy volunteers and homozygous sickle cell disease patients with the addition of excess cholesterol will be performed. Samples from each patient will be treated with excess cholesterol and one of the following: 2-HPBCD, MCD, ApoA1, voxelotor or unsubstituted cyclodextrin. Hemolysis and RBC deformability assays will be performed as described as above. The results of this experiment are expected to show reduced hemolysis and increased RBC deformability upon 2-HPBCD treatment due to extraction of cholesterol from the membrane.

### Example 5

In vitro studies to measure the effect of 2-HPBCD treatment on cholesterol extraction from RBCs from healthy volunteers and homozygous sickle cell disease patients will be performed. Samples from each patient will be treated with one of the following: 2-HPBCD, MCD, ApoA1, voxelotor or unsubstituted cyclodextrin and then RBC ghosts will be generated from the RBC samples. RBC ghosts are RBCs where the hemoglobin has been removed and allows for accurate measurements cholesterol levels in the RBC membrane. Once RBC ghosts are generated membrane cholesterol levels will be measured in each sample. The results of this experiment are expected to show 2-HPBCD treatment resulting in reduced RBC membrane cholesterol levels.

### Example 6

In vivo hemolysis study to measure the effect of 2-HPBCD treatment on hemolysis. Eligible participants will be between 12 and 65 years of age, have confirmed sickle cell disease, have a hemoglobin level between 5.5 and 10.5 g per deciliter during screening, and had 1 to 10 vaso-occlusive crises in the past 12 months. Patients will be treated with 2-HPBCD or placebo, and laboratory markers associated with hemolysis (indirect bilirubin level, absolute reticulocyte count and percentage of reticulocytes, and lactate dehydrogenase level) will be measured from their blood, serum or plasma. Additionally, an annualized incidence rate of vaso-occlusive crisis will be measured in each group. The results of this experiment are expected to show 2-HPBCD resulting in reduced hemolysis in SCD patients based on reduced indirect bilirubin levels, reduced absolute reticulocyte count and percentage of reticulocytes, reduced lactate dehydrogenase levels, and reduced incidence rate of vaso-occlusive crisis.

### EQUIVALENTS

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced therein.

1. A method of reducing membrane cholesterol concentration, improving deformability, and/or reducing hemolysis of a red blood cell (RBC) of a subject suffering from or at risk for sickle cell disease (SCD), comprising contacting the RBC with an effective amount of a cyclodextrin.

2. The method of claim 1 of reducing membrane cholesterol concentration of a red blood cell (RBC) of a subject suffering from or at risk for Sickle Cell Disease (SCD), comprising contacting the RBC with an effective amount of a cyclodextrin.

3. The method of claim 1 or 2, wherein contacting the RBC with an effective amount of a cyclodextrin comprises administering the effective amount of the cyclodextrin to the subject, wherein the subject comprises the RBC.

4. A method of treating or preventing sickle cell disease (SCD) in a subject in need thereof, comprising administering an effective amount of a cyclodextrin.

5. The method of claim 4, wherein the membrane cholesterol levels in red blood cells (RBCs) of the subject is at least 10%, 20%, 30%, 40%, 50%, 60%, 70%, 80%, 90%, 100%, 150%, 200%, 250%, 300%, 400%, 500%, or more, higher than the membrane cholesterol levels in red blood cells (RBCs) of a healthy subject.

6. The method of any preceding claim, wherein the SCD comprises or is sickle cell anemia (SCA).

7. The method of any preceding claim, wherein the subject is homozygous for a genetic mutation that results in the SCD.

8. The method of any preceding claim, wherein the cyclodextrin comprises, or is, a beta (β)-cyclodextrin.

9. The method of claim 8, wherein the β-cyclodextrin is a hydroxypropyl-β-cyclodextrin (HPBCD).

10. The method of claim 9, wherein the HPBCD is a 2-hydroxypropyl-β-cyclodextrin (2-HPBCD).

11. The method of claim 8, wherein the β-cyclodextrin comprises a mixture of 2-hydroxypropyl-β-cyclodextrin (2-HPBCD) molecules.

12. The method of claim 11, wherein the mixture comprises at least 10% 2-HPBCD molecules selected from beta-cyclodextrin substituted with four hydroxypropyl groups, beta-cyclodextrin substituted with five hydroxypropyl groups, beta-cyclodextrin substituted with six hydroxypropyl groups, beta-cyclodextrin substituted with seven hydroxypropyl groups and beta-cyclodextrin substituted with eight hydroxypropyl groups.

13. The method of claim 11 or 12, wherein the mixture comprises less than 1% beta-cyclodextrin substituted with one hydroxypropyl group and less than 1% unsubstituted beta-cyclodextrin.

14. The method of any one of claims 11-13, wherein the mixture comprises at least 10% beta-cyclodextrin substituted with four hydroxypropyl groups.

15. The method of any one of claims 11-14, wherein the mixture comprises at least 10% beta-cyclodextrin substituted with five hydroxypropyl groups.

16. The method of any one of claims 11-15, wherein the mixture comprises at least 10% beta-cyclodextrin substituted with six hydroxypropyl groups.

17. The method of any one of claims 11-16, wherein the mixture comprises at least 10% beta-cyclodextrin substituted with seven hydroxypropyl groups.

18. The method of any one of claims 11-17, wherein the mixture comprises at least 10% beta-cyclodextrin substituted with eight hydroxypropyl groups.

19. The method of any one of claims 11-18, wherein the hydroxypropyl-β-cyclodextrin (2-HPBCD) molecules in the mixture have an average degree of substitution of from about 3 to about 10.

20. The method of any of claims 3 to 19, wherein the administration is intravenous.

21. The method of any of claims 3 to 20, wherein before the administering step, a RBC of the subject exhibits increased membrane cholesterol levels or reduced deformability compared to a healthy subject.

22. The method of any of claims 3 to 21, wherein after the administering step, a RBC of the subject exhibits decreased membrane cholesterol levels or increased deformability, compared to the subject before the administering step.

23. The method of any preceding claim, wherein the method reduces membrane cholesterol levels in the subject, optionally wherein the method reduces excess membrane cholesterol levels.

24. The method of any preceding claim, wherein the method maintains deformability or increases deformability of RBC of the subject.

25. The method of any preceding claim, wherein the method increases RBC deformability in the subject to at least about 5%, at least about 10%, at least about 15%, about 10% to 20%, about 10% to 30%, about 10% to 40%, about 10% to 50%, about 10% to 60%, about 10% to 70%, about 10% to 80%, about 10% to 90% or about 10% to 100% of the deformability of RBCs of a healthy subject.

26. The method of claim 25, wherein the method increases RBC deformability in the subject to at least about 5%, about 10%, about 15%, or about 10% to about 20% of the deformability of RBCs of a healthy subject.

27. The method of any of claims 3 to 26, wherein after the administering step, the deformability of a RBC of the subject is at least about 1.1 fold, about 1.2 fold, about 1.3 fold, about 1.4 fold, about 1.5 fold, about 2 fold, about 3 fold, or about 4 fold greater than the deformability of the RBCs of the subject before the administering step.

28. The method of any preceding claim, wherein the method maintains or reduces hemolysis of the RBC or RBCs of the subject.

29. The method of any preceding claim, wherein the method maintains or increases an average blood hemoglobin concentration and/or RBC number in the subject.

30. The method of any of claims 3 to 29, wherein the method increases an average hemoglobin blood concentration by at least 0.2 g/dL, by at least 0.5 g/dL, by at least 0.7 g/dL, by at least 0.8 g/dL, by at least 0.9 g/dL, or by at least 1 g/dL, relative to a baseline measurement taken in the subject before the administering step.

31. The method of any of claims 3 to 30, wherein the administering step is repeated once daily, once per two days, twice per week, once per week, once per two weeks, once per three weeks, monthly, bi-monthly or yearly.

32. The method of any of claims 3 to 31, wherein the effective amount administered in each dose is about 100 mg/kg to about 6,000 mg/kg.

33. The method of any of claims 3 to 31, wherein the effective amount administered in each dose is about 1 milligrams per kilogram (mg/kg) to about 2,500 mg/kg.

34. The method of claim 32 or 33, wherein the effective amount administered in each dose is from about 500 mg/kg to about 2500 mg/kg.

35. The method of claim 32, 33 or 34, wherein the effective amount administered in each dose is from about 1000 mg/kg to about 1500 mg/kg.

36. The method of claim 34, wherein the effective amount administered in each dose is about 500 mg/kg, about 750 mg/kg, about 1000 mg/kg, about 1500 mg/kg, about 2000 mg/kg, or about 2500 mg/kg, optionally about 1000 mg/kg or about 1500 mg/kg.

37. The method of any of claims 3 to 36, wherein the method comprises repeating the administering step once every two weeks.

38. The method of any of claim 3-30 or 32-36, wherein the method comprising repeating the administering step once daily, once per two days, twice per week, once per week, once per two weeks, once per three weeks, monthly, twice per year, or yearly.

39. The method of any preceding claim, wherein the subject is a human subject.

40. The method of any preceding claim, wherein the RBC or RBCs have increased membrane cholesterol levels, and/or reduced deformability.

41. The method of any of claim 3-5 or 7-40, wherein the subject is suffering from or at risk for Sickle Cell Disease (SCD) is Sickle Cell Anemia (SCA).

42. The method of any one of claims 1-41, wherein the cyclodextrin reduces membrane cholesterol levels, maintains or improves deformability, and/or reduces hemolysis of the RBC or RBCs.

43. Use of an effective amount of a cyclodextrin for a method, wherein the method is as defined by any one of claims 1-42.

44. A cyclodextrin for use in a method of treating or preventing sickle cell disease (SCD) in a subject in need thereof, comprising administering an effective amount of the cyclodextrin.

45. The cyclodextrin for use according to claim 44 wherein the cyclodextrin comprises, or is, a beta (β)-cyclodextrin, optionally wherein the beta (β)-cyclodextrin comprises, or is, a hydroxypropyl-β-cyclodextrin (HPBCD), optionally wherein the hydroxypropyl-β-cyclodextrin (HPBCD) comprises, or is, a 2-hydroxypropyl-β-cyclodextrin (2-HPBCD).

46. The cyclodextrin for use according to claim 44 or 45, wherein the cyclodextrin reduces membrane cholesterol levels, maintains or improves deformability, and/or reduces hemolysis of the RBC or RBCs.

47. The cyclodextrin for use according to any one of claims 44-46, wherein the method is as defined according to any one of claims 1-42.

48. A cyclodextrin for use in a method of reducing membrane cholesterol concentration, maintaining or improving deformability, and/or reducing hemolysis of a red blood cell (RBC) of a subject suffering from or at risk for sickle cell disease (SCD), comprising contacting the RBC with an effective amount of the cyclodextrin.

49. The cyclodextrin for use according to claim 48 wherein the cyclodextrin comprises, or is, a beta (β)-cyclodextrin, optionally wherein the beta (β)-cyclodextrin comprises, or is, a hydroxypropyl-β-cyclodextrin (HPBCD), optionally wherein the hydroxypropyl-β-cyclodextrin (HPBCD) comprises, or is, a 2-hydroxypropyl-β-cyclodextrin (2-HPBCD).

50. The cyclodextrin for use according to any one of claims 48-49, wherein the method is as defined according to any one of claims 1-42.

51. The method of any one of claims 1-3, or of claims 5-42 when dependent from any one of claims 1-3, wherein the method is an ex vivo, in vivo or in vitro method.
